(19) United States
(12) Patent Application Publication
(10) Pub. No.: US 2026/0346027 A1
(43) Pub. Date: Nov. 5, 2026

(54) METHODS AND SYSTEMS FOR IMPLANTING PERCUTANEOUS ELECTRODE ASSEMBLIES FOR PHRENIC NERVE STIMULATION TO TREAT SLEEP APNEA

(71) Applicant: Lunair Medical, Inc., Minneapolis, MN (US)

(72) Inventors: Michael GOFELD, Minneapolis, MN (US); Edward RUPPEL, Minneapolis, MN (US); Mark GELFAND, Minneapolis, MN (US); Zoar ENGELMAN, Minneapolis, MN (US)

(21) Appl. No.: 19/487,213

(22) PCT Filed: May 22, 2024
(86) PCT No.: PCT/US2024/030606

§ 371 Date: Nov. 24, 2025

Related U.S. Application Data
(60) Provisional application No. 63/503,550, filed on May 22, 2023.

(51) Int. Cl. A61N 1/372; A61N 1/05; A61N 1/36

(52) U.S. Cl. CPC A61N 1/372; A61N 1/0558; A61N 1/3601; A61N 1/3605

(57) ABSTRACT

A nerve stimulation lead assembly with an anchoring system includes a lead body with a proximal end configured to connect to an electrical power source and a distal end configured to be positioned adjacent a patient's phrenic nerve. An electrode is positioned at the distal end of the lead body. A first anchor is positioned between the electrode and a terminus of the distal end of the lead body. A sheath encloses the lead body and is axially movable relative to the lead body. The first anchor is biased toward an extended position in which the first anchor extends radially outwardly from the lead body. When the sheath covers the first anchor, the first anchor is kept in a retracted position against a surface of the lead body and when the sheath does not cover the first anchor, the first anchor is in the extended position.

### RELATED APPLICATON

This application incorporates by reference and claims priority to U.S. Provisional application 63/503,550, filed May 22, 2023.

### TECHNICAL FIELD

The field of invention is phrenic nerve stimulation to treat sleep apnea, and particularly, treating sleep apnea by percutaneously implanting an electrode assembly to stimulate the right, left, or both phrenic nerves.

### BACKGROUND

Sleep apnea is a common breathing disorder. A patient suffering sleep apnea repeatedly stops and starts breathing while asleep. These interruptions cause oxygen deprivation in the body through decreased oxygen uptake by the lungs. Sleep apnea often interrupts the patient's sleep cycle, prevents the patient from getting proper rest, and causes the patient to snore loudly. Sleep apnea may increase the risk that a patient will suffer high blood pressure, congestive heart failure and other cardiac related ailments.

Obstructive sleep apnea (OSA) is a common form of sleep apnea in which a patient's airway collapses and prevents air flow to the lungs. Central sleep apnea (CSA) is another form of sleep apnea in which the brain fails to send proper signals to the diaphragm muscle that contracts and expands the lungs to promote breathing.

An approach to treating OSA and/or CSA is to artificially stimulate the phrenic nerve to cause the diaphragm to contract to initiate respiration in a breathing cycle or contract further than would occur during a natural expansion of the lungs. The right-sided phrenic nerve motorically innervates the right part of the diaphragm and the left-sided phrenic nerve motorically innervates the left part of the diaphragm. Sleep apnea treatment may be effective either by unilateral electric stimulation (right or left phrenic nerve) or bilateral electric stimulation (both sides). Artificially stimulating the phrenic nerve may substitute for the brain stimulating the phrenic nerve in the case of CSA. Stimulation of the phrenic nerve may also be effective to treat OSA because contraction of the diaphragm generates mechanical traction on the airway, stiffens and pulls open the airway obstructed due to OSA. Stimulation of the phrenic nerve may also be effective to treat OSA because contraction of the diaphragm generates negative pressure in the airway that may evoke neural reflex to open the airway.

Stimulation of the phrenic nerve to treat sleep apnea has been proposed since no later than the early 2000s, such as in U.S. Patent Application Publications 2003/0195571; 2006/0122662 and 2020/0086118 and U.S. Pat. Nos. 6,415,183 and 7,082,331. To stimulate the phrenic nerve, an electrode assembly is implanted in the patient near the right or left phrenic nerve. The electrode assembly may be surgically implanted through an incision in the neck by using a cervical surgical approach. In contrast, a thoracic surgical approach is through the chest cavity. Thoracic surgery is an invasive surgery creating an opening through the chest cavity and requiring general anesthesia. Nevertheless, in the past more invasive thoracic surgery was preferred because of the challenges of the cervical approach that resulted in frequent device failures.

The anatomy of the patient's neck poses unique challenges that are not experienced when positioning electrodes in other locations. For example, the patient's muscles, bones, blood vessels and nerves are tightly located in the neck and move relative to each other. Such movement can cause an unsecured electrode to move relative to the phrenic nerve. When the electrode moves relative to the phrenic nerve, the impedance of tissues in the path of the electric current may change due to the changing distance between the electrode and the phrenic nerve. Thus, any relative movement between the electrode and the phrenic nerve could affect the current applied to the phrenic nerve and surrounding non-targeted structures. Also, movement of the electrode relative to the phrenic nerve could cause damage to the surrounding vessels and tissue inside the patient's neck, cause pain and discomfort and reduce range of motion. In addition, wires and electrodes made of polymers and metals tend to fatigue and fracture when bent and twisted a lot. All these challenges are well known in the field of cervical phrenic nerve stimulation.

A risk of surgically implanting a nerve stimulation electrode assembly in the neck is that the neck has many nerves, blood vessels, and muscles in close proximity to each other that are at risk of being dissected or damaged during surgery. Also, surgery of the neck may cause painful muscle damage and loss of mobility as the surgeon cuts through muscle to move the electrode assembly into position around or under the peripheral nerve. Additionally, suture-secured electrodes can still move back and forth relative to the nerve when the patient bends or turns their neck.

Surgical placement of an electrode around or adjacent to a phrenic nerve, especially in the neck where the phrenic nerve is under relatively deep tissue and muscle layers and not easily accessible, is risky and difficult. Thus, there is a need for an electrode assembly that is specifically designed to accommodate the cervical phrenic nerve anatomy and a minimally invasive method of surgery to implant the electrode assembly near the phrenic nerve to treat sleep apnea.

Percutaneous implantation methods offer an alternative to surgical methods because they are minimally invasive. Percutaneous methods include the use of a needle to puncture layers of skin, fat, muscle, or other tissues to deliver the electrode assembly proximate to the target nerve. However, migration of permanently implanted electrodes during and after the implant procedure remains a major barrier in the use of chronic percutaneous stimulation systems. Such migration may result in loss of nerve capture, or undesired engagement of nearby excitable tissues. A percutaneous electrode assembly (or implantable lead system) should stay in place during and post-implant procedure for at least 30 days in the changing biological environment of a healing wound before scar tissue buildup makes the implant more secure.

Another major barrier in the use of chronic percutaneous stimulation systems is the limited visibility of the lead assembly once it is positioned.

Percutaneous methods require image guidance, which is typically done by real-time ultrasound (US) imaging but may be assisted by previously acquired magnetic resonance or computed tomography images. A small resection of the skin and subcutaneous tissues can be made to assist percutaneous access, but deep tissue navigation is achieved under US imaging. Tools visible to US imaging are called echogenic. For the purposes of this disclosure, a percutaneous procedure is one where the phrenic nerve is not visualized directly and only non-invasive imaging penetrating through tissue, such as fast real time US and doppler US, is used to gain and secure access to the targeted nerve.

Thus, there is a need for a percutaneous method and system with anchoring mechanisms that will not fail with sudden motion or deteriorate over time. There is also a need for a percutaneous method and system that utilizes implantation components that are echogenic. The current invention is intended to solve these problems in the context of implantable phrenic nerve stimulation for home use by sleep apnea patients.

### SUMMARY

An exemplary method for the percutaneous implantation of electrode assemblies facilitates placement proximate to the phrenic nerve in the neck (cervical approach) to treat sleep apnea. The implantation procedure can be performed as an outpatient procedure. The implantation procedure may be performed with the patient under a local anesthesia, such as using a subcutaneous (SC) administered drug. The method may utilize an introducer kit that includes a sheath, dilator, and a needle may be used to confirm placement adjacent to the phrenic nerve.

In one embodiment, the introducer sheath and dilator are inserted in-plane and placed adjacent to the phrenic nerve. In some embodiments, the needle may have electrodes at the distal tip to provide electrical stimulation. Once the sheath and dilator are in place, a needle having electrodes capable of stimulation is placed inside the sheath and dilator. The stimulating needle is connected to an external pulse generator to deliver electrical pulses. Confirmation of the location of the phrenic nerve may be determined by monitoring contraction of the diaphragm. After confirmation of phrenic nerve location, a percutaneous electrode assembly may be placed.

In another configuration, a hook-like anchor is delivered through a needle or sheath. The hook is mounted on the distal end of a robust wire and the implantable lead system is assembled over the wire.

In another configuration, a separate anchor is inserted on top or over the lead system to prevent migration and add security to the lead positioning. All of these configurations include elements delivered in the collapsed configuration that when expanded to engage surrounding tissues in the neck, such as muscle, fat, fascia, and other connective tissues without damaging or compressing the phrenic nerve.

It may be advantageous to visualize the anchors of the lead system by rendering the anchors echogenic or by placing an echogenic marker adjacent to the anchor(s). In one embodiment, the lead anchors may be made echogenic by modifying the surface or impregnating the anchors with microbubbles to change the echogenicity of the anchors.

The implantable lead system is in electrical communication to a controller such as an implantable pulse generator (IPG) or an external pulse generator (EPG) equipped with an RF link to transfer energy to the subcutaneous antenna, which can be a passive or active antenna. The pulse generator provides the electrical signals transmitted to the implantable lead system and applied to the electrodes in the lead system to stimulate the phrenic nerve. For an IPG, the pulse generator may be surgically implanted in the subclavian pocket, e.g., the infraclavicular region, and the proximal end of the percutaneous implantable lead may be tunneled through tissue, e.g., the prescalene fat pad, to extend from the IPG to the implantable lead.

The implantable pulse generator (IPG) may house a battery and include an antenna to communicate to and receive power from the devices external to the body of the patient. The external devices may include a charger that recharges the battery and a processor configured to receive data from the pulse generator and to transmit, for example, updates to algorithms for stimulating the phrenic nerve. To facilitate communication with and recharging of the pulse generator a patch on the skin, a wand or another wearable device may include antennas and other electronics to relay power and communications between the pulse generator and an external device(s).

The pulse generator may be integrated with or be in communication, e.g., wired or wirelessly, with other devices, such as devices sensing respiration of the patient and body position movements of the patient. Data from these other devices may be used by the pulse generator to determine when and whether or how much to stimulate the phrenic nerve to induce the diaphragm to contract or to contract further than would occur without the stimulation. The sensors integrated into the device can be bioimpedance sensing to detect lung volume, accelerometry to detect motion, and acoustic microphones to monitor breathing.

In another aspect of the technology, a method for percutaneously implanting a device in a living human patient to treat sleep apnea includes using medical imaging to locate the phrenic nerve and other anatomical landmarks, such as the brachial plexus, inserting the introducer sheath and dilator are inserted in-plane and placed adjacent to the phrenic nerve, inserting a needle having electrodes capable of stimulation inside the sheath and dilator, connecting the stimulation needle to an external pulse generator configured to deliver electrical pulses, confirm the location of the phrenic nerve by monitoring contraction of the diaphragm, after confirmation of phrenic nerve location the implantable lead assembly is placed and anchored in surrounding tissues in the neck, such as muscle, fat, fascia, and other connective tissues without damaging or compressing the phrenic nerve. Medical imaging may be used in both pre-planning, such as to plan the path or trajectory and in real time during the procedure to follow the pre-planned trajectory.

In another aspect of the technology, a system for the treatment of sleep apnea in a patient includes a tubular lead system made of a flexible material having at least one pair of conductive stimulation electrodes on the surface of the flexible tubular lead system and a neurostimulation device configured to transfer stimulatory electric current to at least one or one pair (e.g. cathode and anode) of stimulation electrodes; wherein the lead system is configured to be deployed adjacent to the phrenic nerve through the introducer kit comprising a sheath, dilator, and stimulation needle, and wherein the at least one pair of stimulation electrodes is configured to deliver current to activate the phrenic nerve through application of an electric field to a targeted section of the phrenic nerve.

In another aspect of the technology, a system for the treatment of sleep apnea in a patient by stimulation of the phrenic nerve delivers pulse trains of electric field energy to the targeted nerve location during inspiration and expiration periods at a set breathing rate where both periods of stimulation force diaphragm contraction in excess of unstimulated state and where the inspiration period energy is higher than the expiration period energy.

### SUMMARY OF FIGURES

The figures in this application illustrate exemplary percutaneous implantable lead systems and an exemplary approach to percutaneous implantation of the implantable lead systems to treat sleep apnea, the figures are:

FIG. 1 illustrates a patient undergoing a percutaneous procedure to implant a lead system in the patient's neck.

FIG. 2A illustrates a cut-away view of a patient's neck showing the internal anatomy of the neck.

FIG. 2B is a cross-sectional view of the neck to show the internal anatomy of the neck at the C7 level.

FIG. 3 illustrates a cross-sectional view of the neck showing the fascia of the neck.

FIGS. 4A-4C show ultrasound images of the internal anatomy of the neck.

FIGS. 5A-5E illustrate an exemplary method for percutaneously implanting a lead system.

FIG. 6 is a flow chart of a stepwise method for over-the-wire percutaneous implantation of a lead system.

FIG. 7 illustrates an imaging system and sensor system useful in implanting a lead system into the neck of a patient.

FIGS. 8A and 8B are views of the proximal end of exemplary lead systems.

FIG. 9 is an illustration of the distal end of a lead system having tines at the distal tip overlying the phrenic nerve.

FIGS. 10 and 11 are illustrations of the distal end of a lead system having a hook at the distal tip.

FIG. 12 illustrates a lead system having an electrode assembly and tines at the distal tip overlying the phrenic nerve. The lead system includes a locking mechanism to affix an anchoring mechanism at the distal end of the electrode assembly.

FIGS. 13A and 13B show the distal end of a lead system before and after removal of the sheath to expose the stimulation electrodes with a hook and barbs at the distal tip for fixation where the lead system is overlying the phrenic nerve.

FIG. 14 illustrates a patient with an implanted electrode and implanted pulse generator (IPG) and an external controller that interacts with the IPG and communicates with doctors via a cloud computer system.

FIG. 15 is a block diagram of an exemplary IPG.

FIG. 16 is a schematic diagram of a circuit board of the IPG.

### DETAILED DESCRIPTION

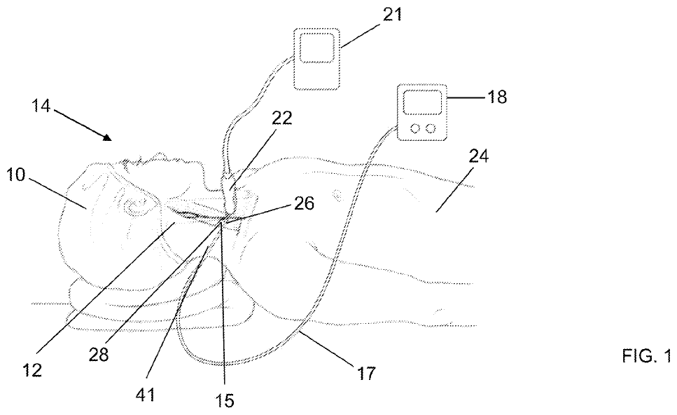

FIG. 1 shows a patient 10 undergoing a percutaneous procedure to implant an electrode assembly in the patient's neck 12. FIG. 1 also shows a system that is used to insert, position, test, fixate, and implant a phrenic electrode assembly 15 in the patient's neck 12. The percutaneous lead 41 may include an electrode assembly 15 mounted on or introduced via an insertion tool that can be, for example, a sharp needle, a blunt needle, or a cannula. The electrode assembly 15 may include one or more electrodes and a stimulation lead 41. The length of the stimulation lead 41 may be 25 to 45 mm.

During implementation, a proximal end of the lead 41 may be electrically connected to a temporary external pulse generator (EPG) 18 via a lead extension 17 The EPG 18 can create a series of short electric pulses that are known to cause action potentials in a motor nerve (pulse trains). A sterile segment of the lead extension 17 may connect to another segment of the lead extension 17 that is non-sterile and connects to the non-sterile EPG 18.

Alternatively, the electrode assembly 15 may be in wireless communication with the EPG 18.

The EPG 18 may generate a test stimulation signal to confirm that the electrode assembly 15 is positioned proximate to the phrenic nerve 28.

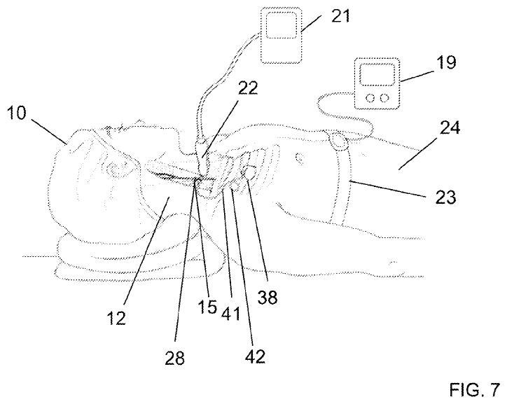

Placement is confirmed by visually observing the contraction of the patient's abdomen 24 or observing feedback signals indicative of the contraction of the patient's diaphragm that are generated by a wearable monitoring system 23 (FIG. 7) placed on the patient's abdomen 24. Contraction of the patient's diaphragm indicates that nerve capture (of the phrenic nerve 28) has been achieved.

Diaphragmatic twitches or contractions observed when the stimulation current is less than 5 mA (or preferably less than 2 mA) indicates that the stimulating cathode electrode of the electrode system 15 is within several millimeters from the nerve. However, if nerve capture is lost or the stimulation current needed to contract the diaphragm becomes too high (e.g., 2 mA or greater or 5 mA or greater), the electrode is out of position and needs to be repositioned.

A diaphragmic twitch or contraction detected by a sensor (e.g., accelerometer) on the patient's abdomen 24 or chest at exactly the expected time corresponding to the time when energy was applied may be an indication of nerve capture and can be transmitted to the operator by way of a visual signal or auditory beep. For example, pulse trains of stimulation pulses of 2 mA can be applied automatically 20 times a minute to generate rhythmic twitches. The twitches or contractions suddenly stopping or the signal indicative of the twitches or contractions being lost may indicate that the electrode has migrated from the desired location.

FIG. 1 also illustrates an anterior cervical puncture site 26 (access point) that is suitable to percutaneously form a passage through natural tissue and tissue interfaces in the patient's neck 12 to access at least one of the phrenic nerves 28 (left or right). The percutaneous implantation system 14 may also include a fixation tool (not shown) to secure and immobilize the electrode assembly 15 in the patient's neck 12 as well as an ultrasound imaging device 21 with an ultrasound probe 22 applied to the patient's neck 12.

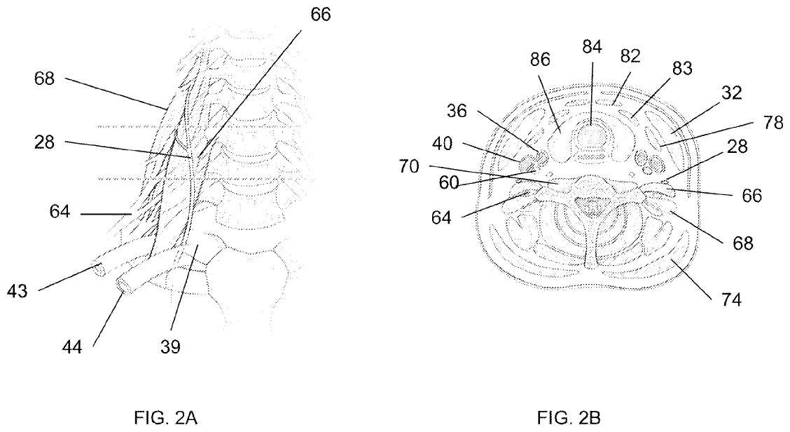

FIG. 2A illustrates a cut-away view while FIG. 2B is a transverse slice of a patient's neck 12 showing the internal anatomy of the neck 12. As can be seen, some structures in the patient's neck 12 are omitted from FIG. 2A in order to emphasize the target portion of phrenic nerve 28. The target portion of the phrenic nerve 28 may be at the C7 vertebral level. The neck anatomy is dense with nerves, major blood vessels, muscles, and other internal organs. The patient's neck 12 includes major blood vessels such as the internal jugular vein 36, the carotid artery 40, the subclavian artery 43 and the subclavian vein 44. In addition to the phrenic nerve 28, the neck 12 includes the vagus nerve 60 and the brachial plexus nerves 64. Muscles in the neck include the sternocleidomastoideole 32, the anterior scalene muscle 66, the middle scalene muscle 68, the longus colli muscle 70, the trapezius muscle 74, the omohyoid muscle 78, the sternohyoid muscle 82, and the sternothyroid muscle. The neck 12 also includes the trachea 84 and thyroid 86. The presence of these blood vessels, nerves, and muscles in the neck 12 create obstacles to access the phrenic nerve 28 and implant the electrode assembly 15.

Anatomical structures in the neck 12 that can be particularly problematic when accessing the phrenic nerve 28 include the subclavian artery 43 and the subclavian vein 44 on the inferior (lower) side of the phrenic nerve 28. On the anterior side of the phrenic nerve 28, the structures within the carotid sheath can also be problematic. These structures include the internal jugular vein 36, the carotid artery 40, and the vagus nerve 60. Superior-laterally relative to the phrenic nerve 28, the brachial plexus nerves 64 can also be problematic. The brachial plexus nerves 64, derived from C5-C8 fibers, leave the neck region between the anterior and middle scalene muscles 66, 68.

The phrenic nerve 28 is located on the anterior surface of the anterior scalene muscle 66, while the brachial plexus nerves are located on the posterior side of the anterior scalene muscle 66. Thus, the anterior scalene muscle 66 intervenes between the brachial plexus nerves 64 and the phrenic nerve 28 at the targeted level. Accordingly, the location of the phrenic nerve 28 on the anterior scalene muscle 66 allows the phrenic nerve 28 to be accessed from an anterior cervical location while safely avoiding the brachial plexus nerves 64 so that the brachial plexus nerves 64 are not damaged and/or unintentionally stimulated during apnea therapy.

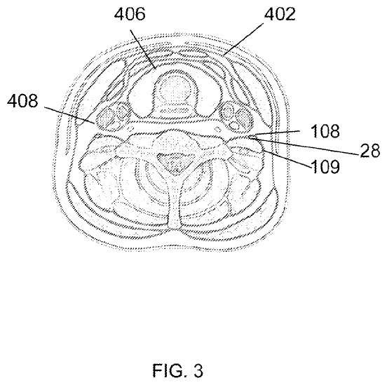

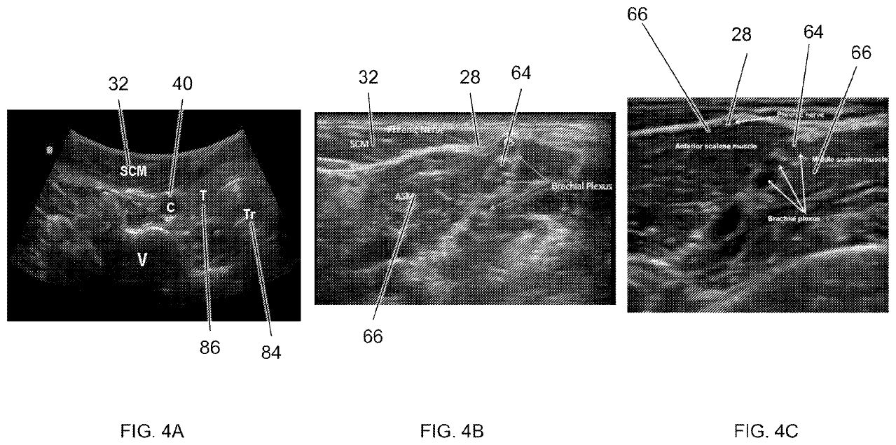

FIGS. 3-4C show the internal anatomy of the patient's neck 12. FIG. 3 shows various fascias (or sheaths) in the neck 12. A fascia is a thin sheet of connective tissue, which is primarily collagen, and functions to stabilize, enclose, and separate muscles and internal organs. The phrenic nerve 28 is a bundle of nerves enclosed in a fascia or sheath 108. The prevertebral fascia 109 separates the anterior scalene muscle 66 from the phrenic nerve sheath 108. It should be understood that any reference to a nerve includes the nerve bundle and the sheath (or fascia).

A deep cervical fascia acts to compartmentalize most structures of the neck and prevents the spread of infections. It consists of three fascial layers (or sheaths), which are the superficial layer 402, the pretracheal layer 406, and the prevertebral layer 109. These layers of the deep cervical fascia also function to support the viscera of the neck (e.g. thyroid gland), muscles, blood and lymphatic vessels, and deep lymph nodes. They also form the carotid sheath 408 that surrounds the common carotid arteries 40, internal jugular vein 36, and the vagus nerve 60. The superficial layer 402 of the deep cervical fascia surrounds the whole neck 12 and contains the trapezius 74 and sternocleidomastoid muscles. The pretracheal fascia 406 forms a natural boundary between the muscles in the neck 12 behind the trachea 84. The prevertebral layer 109, as the name implies, forms a sheath for the vertebral column and the muscles (such as the longus colli 70) associated with the vertebral column.

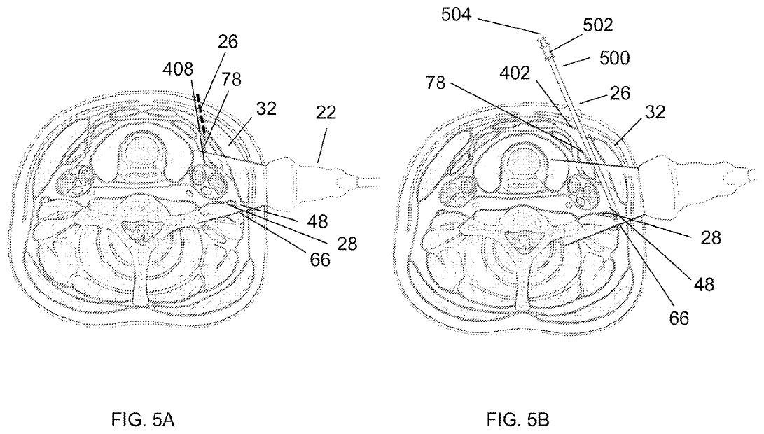

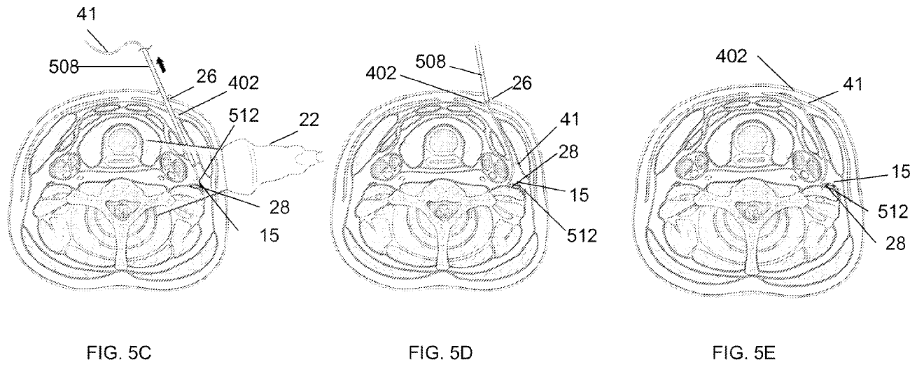

FIGS. 5A-E illustrate a method for percutaneous implantation of the electrode assembly 15 proximate to the phrenic nerve 28. It is understood that there are many useful tools available for a skilled professional to execute this procedure and the choice of technique is often different for different operators. It is envisioned that in some cases an insertion tool 502 may be inserted into the patient's neck 12 to create a path to the target phrenic nerve 28 and then a guidewire may be inserted into the insertion tool. The insertion tool may be exchanged for a dilator, which may be provided over the guidewire. In other embodiments the actual implanted lead may serve as a dissection tool, reinforced by a removable stylet. Regardless of the technique a preplanned path is followed using real time ultrasound imaging. This path follows the boundary separating two major muscles: anterior scalene and sternomastoid. The path is planned to avoid major blood vessels and nerves such as brachial plexus. Electric stimulation may be used to confirm that the electrodes are in the right position before and after the expandable securement features are employed to prevent migration before the permanent lead is routed to the pulse generator with the appropriate strain relief to compensate for the motion of the neck.

FIG. 5A illustrates the selection of the location of a cervical puncture site 26. The location of the cervical puncture site 26 may be selected based on how the phrenic nerve 28 is to be accessed. For example, the phrenic nerve 28 may be accessed using an anterior approach as shown in FIGS. 5A-E. It is also contemplated that the phrenic nerve 28 may be accessed using a posterior approach. The initial puncture may be limited to superficial tissue layers to expose the entry point (e.g., the edge of sternomastoid muscle).

To locate the puncture site 26 and plan the pathway from the entry point to the phrenic nerve 28, the ultrasound probe 22 may be used to locate the phrenic nerve 28, other nerves, muscles, and major blood vessels (FIG. 5A). An ultrasound image may be obtained before the puncturing the patient's skin to plan the path to the target phrenic nerve 28. The ultrasound probe 22 may then be used during the procedure to guide the advancement of echogenic instruments along the pre-planned path. FIG. 5A illustrates planning of the implantation pathway. The operator of the ultrasound device 21 may apply the ultrasound probe 22 to the neck 12 and the device 21 may generate on a display, images of internal organs and tissue boundaries designated as echogenic landmarks in the neck 12 (see FIGS. 4A-C).

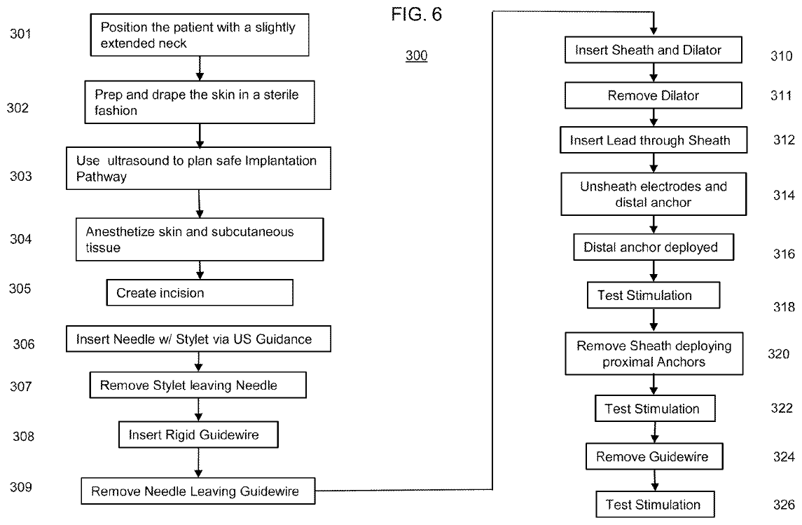

FIGS. 5A-5E and the flow chart in FIG. 6 illustrate a method for preparing a patient for percutaneously implanting an electrode assembly 15 and then percutaneously implanting the electrode assembly 15. First, the patient may be positioned with a slightly extended neck (step 301). Then, the patient's skin may be prepared and draped in a sterile fashion (step 302). Next, a physician may use an ultrasound device 21 to plan a safe implantation pathway (step 303) by locating the phrenic nerve 28 and other anatomical landmarks such as brachial plexus 64, thyroid gland 89, carotid artery 40, internal jugular vein 36, sternocleidomastoideole 32, anterior scalene muscle 66, and the middle scalene muscle 68. The facia that separates sternocleidomastoideole 32 and anterior scalene muscle 66 may define or bound the pathway to the phrenic nerve. Alternatively, the physician can use a real-time MRI/Ultrasound Fusion navigation system to locate the phrenic nerve 28 and anatomical landmarks. The MRI image of the neck 12 may be pre-acquired and digitally combined with the US image in real time. A surgeon may prefer to use a blunt dissection instrument that separates rather than punctures the tissue layers along their natural boundaries.

Once the safe implantation pathway is planned, the patient's skin and subcutaneous tissue may be anesthetized (step 304) and puncture site 26 may be created (step 305). Once the puncture site 26 is created, a delivery system 500 may be inserted through the puncture site 26 along the planned path under ultrasound guidance (FIG. 5B) (step 306). The superficial fascia 402 may be pierced or resected and the delivery system 500, which may include an insertion tool 502 with a stiffening stylet 504 inside. The insertion tool 502 may be a sharp needle, a blunt needle, or a blunt cannula and may be made of a polymer or combination of braided metal and polymer layers and may have a diameter of 0.7 to 1.3 mm. It is contemplated that the insertion tool 502 may be stiffened by the stylet 504 and advanced until the distal end of the insertion tool 502 is positioned proximate to the phrenic nerve 28 by pushing the insertion tool 502 along the natural muscle separation boundary visible on an ultrasound monitor as a reflective facia that separates anterior scalene and sternomastoid muscles. The distal tip of the insertion tool 502 may be conductive or equipped with electrodes to allow an electrical current to be delivered through the distal tip.

The delivery system 500 may be inserted under US guidance. Once the delivery system 500 is in the desired target location based on US visualization, the stylet 504 may be removed leaving the insertion tool 502 in place (step 307). Then, a stiff guidewire 506 is inserted into the lumen of the insertion tool 502 until the guidewire 506 extends slightly past the distal tip of the insertion tool 502 (step 308). The insertion tool 502 may now be removed leaving the guidewire 506 in place (step 309). The guidewire 506 may be used as part of an over-the-wire delivery system for the electrode assembly 15. Next, a sheath 508 and dilator 510 may be inserted over the guidewire 506 (step 310). The dilator 510 may create a pathway from the skin to the target site proximate to the phrenic nerve 28. The dilator 510 may then be removed (step 311). Once the dilator 510 is removed, the electrode assembly 15 may be inserted over the guide wire 506 through the sheath 508 (step 312)

FIG. 5C illustrates partial removal of the sheath 508 to uncover a distal electrode array 516 and one or more distal anchors 512 (step 314). The distal anchor 512 may then be deployed (step 316). The electrode assembly 15 may be connected to an external pulse generator (stimulator) 18 (either directly or by way of a lead extension 17) and a test stimulation may be performed (step 318). Successful placement of the electrode assembly 15 may be confirmed by visually and/or audibly observing indicators of a contraction of the diaphragm. An unintended contraction of muscles in the back or in the chest may indicate undesired proximity of motor nerves that should not be stimulated such as nerves of the brachial plexus or ancillary nerve.

Once a contraction of the diaphragm is observed, the sheath 508 may be further removed (FIG. 5D), thereby deploying the proximal anchors 514 (step 320). Another test stimulation may then be performed to confirm that the electrode assembly 15 remains correctly located (step 322). Confirmation may be made by visually and/or audibly observing indicators of a contraction of the diaphragm. An unintended contraction of muscles in the back or in the chest may indicate undesired proximity of motor nerves that should not be stimulated such as nerves of the brachial plexus or ancillary nerve. Once it is confirmed that the electrode assembly 15 is correctly located, the guide wire 506 may be removed (step 324) and a final test stimulation may be performed (step 326).

FIG. 5E illustrates the cross-sectional view of the electrode assembly 15 after implantation and after the puncture site 26 is closed. The anchor(s) 512 and proximal anchor(s) 514 may retain the electrodes proximate to the targeted phrenic nerve section after the site is fibrosed and scar tissue has been formed that further improves the resistance to migration.

After the electrode assembly 15 is properly placed near the phrenic nerve 28 and secured to the surrounding anatomical structures, the lead 41 may be detached from the delivery system 500 and the external pulse generator 18 and may be electrically coupled to a pulse generator that may be an IPG 38 implanted in the patient's body, or an external wireless pulse generator positioned next to the patient's neck 12 while the patient sleeps.

Following the final percutaneous lead placement and removal of the sheath 508, a tunneling tool may be used to tunnel the lead 41 to the pectoral region where the IPG 38 is implanted. Tunneling the lead 41 involves passing the lead 41 under the skin over the collar bone to a subcutaneous pocket in the chest or armpit where the IPG 38 is implanted. Once the pocket for the IPG 38 is created, the IPG 38 may be connected to the lead 41. The system may be tested using a unique protocol within the programming device to ensure diaphragm contraction upon test stimulation delivery before the final step of suturing the skin where the IPG 38 is implanted.

FIG. 7 is an illustration that shows a patient implanted with a percutaneously delivered permanently implanted phrenic nerve stimulation system. The lead 41 is shown equipped with a strain relief 42 and tunneled to the subclavian pocket implanted IPG 38. Multiple strain relief components may be incorporated or assembled with the lead 41 before or after implantation. Such strain relief components may be suture loops or a portion of the lead 41 adapted to receive a tack or a staple. The purpose of the strain relief 42 is to limit the potential migration of the lead 41 along its track under the patient's skin. A respiratory sensor (part of the wearable monitoring system 23) may be mounted to the patient's chest or abdomen 24 to measure the patient's diaphragmatic contractions. It may be, for example, a motion sensor, a bioimpedance sensor, or an accelerometer. The sensor may transmit data to a monitor 19. It is contemplated that the IPG 38 may be in communication with external devices. A user interface of the monitor 19 may notify the user of the breathing activity and diaphragmatic contractions and can be manipulated by the user to actuate the IPG 38 so that the IPG 38 delivers electrical current pulse trains to the electrode assembly 15 to stimulate the phrenic nerve 28. Alternatively, the IPG 38 may automatically deliver electrical current pulse trains to the electrode assembly 15 to stimulate the phrenic nerve 28 in response to sensor signals indicative of the patient's breathing activity. The electrical current pulse trains may be delivered as therapy to treat the patient's sleep apnea.

It is understood that there are many possible configurations of implantable devices such as IPGs 38, leads 41, EPGs 18 and wireless stimulation devices. There may even be completely leadless devices small enough to reside in the neck 12 of the patient near the phrenic nerve 28 in their entirety. Common to all these solutions, they all include electrically conductive electrodes, and it is important to prevent migration of stimulation electrodes in their relation to the phrenic nerve 28. It is generally desired to maintain stimulation current between 1.0 and 5.0 mA and distance between the electrode and the nerve between 0.5 and 5 mm.

The electrode assembly 15 may include one or more stimulation electrodes and the lead 41. The lead 41 may have a tubular shape that is implanted permanently in the space or track initially created in the patient's neck 12 by the insertion tool 502 and possibly dilated by other devices. The distal end of the lead 41 is equipped with stimulation electrodes that may be ring electrodes. The body of the lead 41 may be made of a polymer like urethane or silicone. The walls of the lead 41 may be reinfused by braid.

Separate conductive wires that conduct electric signals may be located inside the walls of the lead 41. It is contemplated that the lead 41 may be a tubular, coaxial assembly of conducting and non-conducting components with a lead body extending between a distal end and a proximal end, and the distal end having at least one electrode of an electrode array extending longitudinally from the distal end toward the proximal end. The lead body at its proximal end may be coupled to the IPG 38, an EPG 18, an additional intermediate wiring such as an antenna for wireless energy transfer, or another stimulation or monitoring implantable device.

The lead 41 may have a central lumen for a stylet or a guidewire that gives the lead 41 more structural stability and stiffness to facilitate the insertion of the lead 41 through a channel in the insertion tool 502. The lead 41 may also have a consistent outer diameter along its length to assist insertion through the channel in insertion tool 502. In addition, the lead 41 may be configured so that the lead 41 may flex with the motion of the patient's neck 12 but never fracture or break.

The electrode assembly 15 may also include a lead securement device. The lead securement device may be in the form of an anchor. The purpose of the lead securement device is to immobilize the electrodes on the electrode assembly 15 by securing the electrode assembly 15 to the anterior scalene muscle 66, sternocleidomastoideole 32, other muscles, facias, and/or connective tissues in the vicinity of the phrenic nerve 28. The securement device is not intended to penetrate, compress, or engage the phrenic nerve itself directly.

The intention behind the securement is that the distance between the electrodes of the electrode assembly 15 and the phrenic nerve 28 remains relatively constant. That is the securement of the electrode assembly 15 to the surrounding tissue prevents or limits the migration (e.g., axial movement) of the electrodes of the electrode assembly 15 due to a manipulation by the physician or due the patient moving their head or neck.

It has been discovered that electrode leads are most susceptible to migration in the first 30 days after being implanted, but patients cannot be expected to move much less than they normally do. In the first days after implantation, inflammation and disrupted cells fill the cavity surrounding the lead 41 with exudate and blood and the cavity may expand. Later on, what is often described as a “capsule” or channel like “track” may form within which the lead 41 may move axially. It is desired to have an anchor that will prevent migration in the acute scenario during the insertion procedure and during the healing process that will also fibrose well and integrate into the surrounding tissues later.

The securement device may be one of an array of securement devices or one of several securement devices of different types. The securement device may be integral to the lead 41 or delivered separately and affixed to the lead 41 during the implantation procedure. The securement device may be partially or completely made of a metal, such as nitinol or stainless steel, of a non-biodegradable polymer such as nylon or of a biodegradable polymer that will gradually dissolve after the implanted lead is securely fibrosed in place. It may also comprise a combination of biocompatible materials.

In one form, the securement device may include polymer tines. Such polymer tines may be designed so that when the polymer tines are pushed into the tissue they collapse and when the polymer tines are pulled back they resist and bite into the surrounding tissue. The polymer tines may also be designed to collapse when a pulling force acting on the polymer tines is above a threshold. Accordingly, polymer tines may provide a method to both temporarily secure the electrode assembly 15 to the tissue surrounding the phrenic nerve 28. For example, if it is desired to move the electrode assembly 15 to another location after the polymer tines have already been deployed, the lead 41 may be firmly pulled so that the force acting on the polymer tines is above the threshold force. Such a force will cause the polymer tines to collapse and possibly invert, thereby allowing the electrode assembly 15 to be moved to another location.

Tines made out of metal such as nitinol may provide necessary resilience and strength of attachment in the patient's neck area. It is appreciated that the more resilient metal tines may not be extractable without surgery.

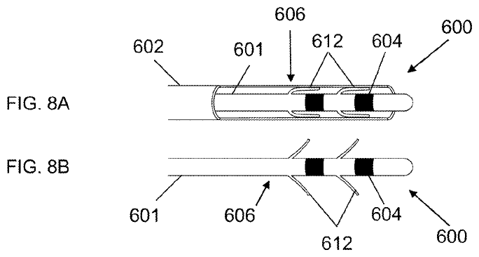

One approach to permanently anchoring the implanted electrode assembly is shown in the electrode assembly 600 of FIGS. 8A and 8B. The electrode assembly 600 may include a stimulation lead 601 contained within a removable sheath 602. An array of stimulation electrodes 604 may be located on or embedded within a surface of the stimulation lead 601 at a distal end. A securement device 606 (single-tine or micro-barb) may be integrated into the distal end of the stimulation lead 601. In some embodiments, double-tines or micro-barbs may be integrated into the stimulation lead 601 at the distal end of the stimulation lead 601. The securement device 606 may include one or more anchors 612 (e.g., tines). The anchors 612 may be adjacent one electrode 604 or may be between two electrodes 604. It is contemplated that the anchors 612 and the electrodes 604 may be positioned in an alternating pattern as shown in FIGS. 8A and 8B. In addition, an anchor 612 may be between the terminal end of the stimulation lead 601 and an electrode 604. Alternatively, an electrode 604 may be between an anchor 612 and the terminal end of the stimulation lead 601. In addition, each anchor 612 may have one or more tines positioned radially around the stimulation lead 601. FIGS. 8A and 8B show a configuration in which each anchor 612 comprises two tines on opposite sides of the stimulation lead 601. However, any radial pattern may be used.

As can be seen in FIG. 8A, when the sheath 602 covers the anchors 612, the anchors 612 may be pressed against the surface of the stimulation lead 601 in a retracted position so that the anchors 612 are prevented from engaging any tissue around the phrenic nerve. Thus, while the sheath 602 covers the anchors 612, the stimulation lead 601 is movable relative to the phrenic nerve 28.

Each anchor 612 may be flexible and pliant with a width and thickness and extending through a tine length from an attached tine end to a free tine end. In addition, each anchor 612 may have an effective diameter of about 2-3 French units. The attached anchor end is attached to the body of the lead 601 from an anchor attachment site and supports the anchor 612 extending outwardly of the body of the lead body 601 and proximally toward the lead proximal end. The anchors 612 may be adapted to be folded inward against the body of the lead 601 when fitted into and constrained by the lumen of the sheath 602 such that the anchor free ends of more distal anchors 612 are urged toward or alongside the attached anchor ends of the adjacent more proximal anchors 612, and the folded anchors 612 do not overlap one another.

The anchors 612 may be biased to an outwardly extended position (spring activated) so that when the sheath 602 is removed from the anchors 612, the anchors 612 are allowed to spring outward to a deployed position (FIG. 8B). In the deployed position, the anchors 612 may engage the tissue surrounding the phrenic nerve 28 to lock the stimulation lead 601 in position relative to the phrenic nerve 28. In addition, the anchors 612 may be deployed one at a time as the sheath 602 is slid along the stimulation lead 601. For example, during positioning and testing, the sheath 602 may be moved a distance that only deploys one of the anchors 612 while keeping the second anchor 612 retracted. When the sheath 602 is only partially removed and only one anchor 612 is deployed, the stimulation lead 601 may be locked in position relative to the phrenic nerve 28 but may still be allowed to pivot around the deployed anchor 612.

Although FIGS. 8A and 8B show the anchors 612 being oriented so that the free end of the anchors 612 are oriented toward the distal end of the electrode assembly 600, it should be understood that the anchors 612 may have the opposite orientation (i.e., the free end of the anchors 612 may be oriented toward the proximal end of the electrode assembly 600. In other configurations, the free ends of some anchors 612 may be oriented toward the distal end of the electrode assembly 600 while the remaining anchors 612 are oriented toward the proximal end of the electrode assembly 600. In addition, the anchors 612 should be located so that the anchors 612 do not engage or make contact with the nerves when deployed.

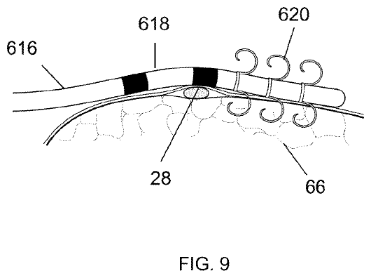

FIG. 9 illustrates a lead 616 with curling tines 620 that are made from a shape memory alloy. When released from the sheath 602, the tines 620 may assume their curvature back towards the proximal end of the lead 616, thereby increasing the resistance to axial misalignment of the lead electrodes 618 in relation to the phrenic nerve 28. The tines 620 may become firmly embedded in the muscle 66 and fascia covering the muscle immediately after deployment and before the process of fibrosis and healing are completed.

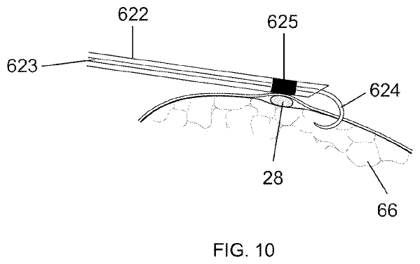

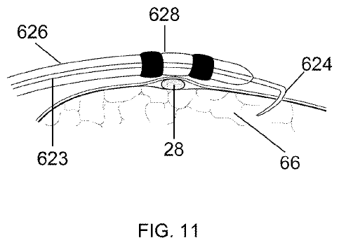

It is contemplated that a permanent anchor (an anchor that is not removable without surgery) may be incorporated in the lead design. Such an anchor may be robust enough to prevent migration even with extreme motion and muscle strain. FIGS. 10 and 11 illustrate an exemplary configuration with a permanent anchor.

The permanent anchor configuration may be implanted using the method disclosed above and illustrated in FIGS. 5A-6. FIG. 10 shows a stage of the implantation procedure in which an insertion tool 622 has been inserted along the implantation pathway and is positioned adjacent the phrenic nerve 28. The insertion tool 622 may be a sharp needle, a blunt needle, or a blunt cannula. In addition, the insertion tool 622 may include an electrode 625 adjacent the terminal end of the insertion tool 622. The electrode 625 may be connected to the external pulse generator 18 (or IPG 38) by way of the insertion tool 622 and a lead or wire (not shown). The electrode 625 may allow the user to determine whether the insertion tool 622 is in the desired location. For example, a stimulation signal from the external pulse generator 18 (or IPG 38) may be actuated and a stimulation signal may be sent to the electrode 625. The patient may be monitored to determine whether the electrode 625 is in position to actuate the phrenic nerve 28.

A guidewire 623 may be inserted through a lumen of the insertion tool 622. The guidewire 623 may terminate at an anchoring device 624. The anchoring device 624 may be in a shape that resembles a barb or a fishhook and may be designed to engage tissue on a distal side of the phrenic nerve 28. Given that the desired location and orientation of the insertion tool 622 and the guidewire 623 crosses the phrenic nerve 28 orthogonally, the anchoring procedure may take advantage of the specific anatomical location where the placement of an electrode lead 626 is optimal (i.e., the location where the freedom of motion within the neck is minimal and other nerves are the furthest away from the phrenic nerve 28).

The electrode assembly 625 may be positioned over the phrenic nerve 28 between the anterior surface of the anterior scalene muscle 66 and the prevertebral fascia of the deep cervical fascia. Alternatively, the electrode assembly 625 may be positioned over the phrenic nerve 28 and the fascia, anchored to the anterior surface of the prevertebral fascia 109 and between the anterior surface of the prevertebral fascia 109 and the posterior surface of the sternocleidomastoideole 32.

To lock the electrode assembly 625 in place once positioned in the desired location, the anchoring device 624 may be pushed out of the lumen of the insertion tool 622 by pushing the guidewire 623 through the lumen of the insertion tool 622. It is contemplated that the anchoring device 624 may curve as it exits the insertion tool 622. The anchoring device 624 can be retracted back into the insertion tool 622 for repositioning by pulling on the guidewire 623.

When the anchoring device 624 is deployed out of the insertion tool 622, the anchoring device 625 may curl back toward itself in a hook shape.

Accordingly, anchoring device 624 can be firmly secured (or hooked) to the adjacent muscle (e.g., anterior scalene muscle 66) by pulling back on both the insertion tool 622 and the guidewire 623. By performing this maneuver, the anchoring device 625 can be pulled back without being retracted back into the insertion tool 622. In addition, the insertion tool 622 can be torqued, rotated around the axis, to achieve the desired orientation with the anchoring device 624 exiting towards the anterior scalene muscle 66 and prevertebral fascia 109 to secure the assembly to the muscle and fascia. In order to maintain the desired orientation of the insertion tool 622 and the anchoring device 624, the lumen of the insertion tool 622 may be radially asymmetric to prevent the anchoring device 624 from rotating inside the lumen.

After the anchoring device 624 is firmly embedded within the tissue adjacent to the phrenic nerve 28 and the position for the final implantation of electrodes is confirmed by electric stimulation, the insertion tool 622 may be withdrawn leaving the guidewire 623 in place. The permanent lead 626 with the electrodes 628 may be introduced over the guidewire 623 so that a central lumen of the permanent lead 626 is slidably engaged with the guidewire 623. See FIG. 11.

As shown in FIG. 11, the lead 626 is pushed forward distally while the guidewire 623 is held tight with the anchoring device 624 deeply embedded in the tissue distal of the crossing of the nerve 28. The operator can immobilize the guidewire 623 at the proximate end of the lead 626 with a screw or other tightening mechanism to prevent future migration of the lead 626 over the wire 623. The excess portion of the guidewire 623 extending beyond the proximate end of the lead 626 may be clipped and trimmed. The guidewire 623 and the lead 624 may become a sealed lead body assembly. The electrical and mechanical terminus of the lead 626 at the proximate end can be connected to a lead extension 17 tunneled to the receiving header of the IPG 38 or subcutaneous antenna implanted in a subcutaneous pocket. Alternatively, the lead 626 may be directly attached to the IPG 38 or subcutaneous antenna.

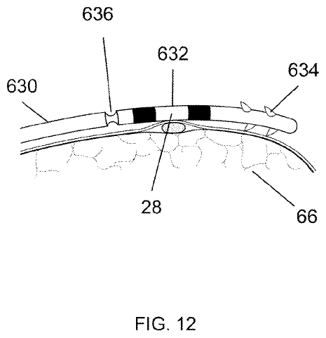

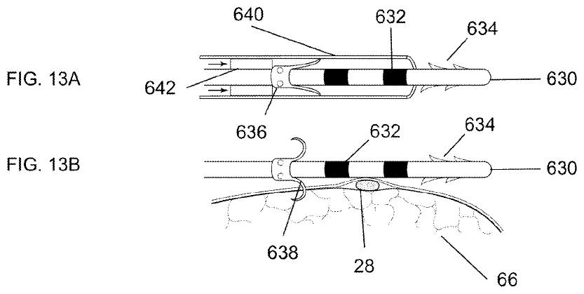

In another embodiment, a lead retention anchor may be introduced after the lead is positioned. The anchor may be pushed out over the lead from a sheath. This method may require larger devices, dilation, and an exchange of tools. As shown in FIGS. 12-13B, a lead 630 may be equipped with anchors 634 sufficient to hold the lead 630 in place and prevent migration during the procedure. The anchors 634 may be in the form of collapsible polymer tines.

The lead 630 may be equipped with a locking mechanism 636 (FIG. 12) adapted to secure an additional fixation mechanism slidable along the lead body. The locking mechanism 636 may be a groove, a stop or a small taper designed to jam and immobilize the fixation mechanism when it is pushed along the lead 630 from the proximal to distal end.

The lead 630 may be positioned over the phrenic nerve 28 and it may be stiffened by an internal stylet or guidewire. A sheath 640 may be introduced over the lead 630 from the proximal end adapted to receive a fixation crown anchor 638 (FIGS. 13A, 13B). The anchor 638 can be pushed along the length of the lead 630 inside the sheath 640 by a pusher or pushing tube 642. The pushing tube 642 may engage the locking mechanism 636 that gives resistance feedback to the operator and prevents further motion of the anchor 638 in either a proximal or distal direction. The sheath 640 and the pusher 642 may be removed by being slid toward a proximal end of the lead 630. As the sheath 640 and the pusher 642 are removed, the anchor 638 may expand into a fixation position with prongs biting into the surrounding prevertebral fascia 109, muscle 66, and/or connective tissue.

When the pusher 642 and the sheath 640 are withdrawn and the anchor 638 is released, nitinol prongs of the anchor 638 may spring out and penetrate muscle and/or fascia (FIG. 13B). The locking mechanism 636 may include and/or be in the form of a stopper groove and may be incorporated in the body of the lead 630 to secure the anchor 638. The anchor 638 may be positioned distal or proximal of the electrodes 632 or between the electrodes 632. More than one anchor 638 can be used. The locking mechanism 636 may be designed to break if the lead 630 is yanked back with a force that is above a threshold. For example, if there is an emergency that requires the lead 630 to be removed, the locking mechanism can be overcome by pulling on the lead 630 with a force that exceeds a threshold. In this way the anchor 638 may remain in-grown in the tissue and the lead 630 can be pulled out of the track.

In some instances, the components of the percutaneous lead delivery system and/or lead, may incorporate changes to increase the visibility using imaging techniques. The use of ultrasound during placement of percutaneous lead systems is prevalent, thus it is advantageous to make elements of the lead system easier to visualize using ultrasound (or echogenic). An echogenic percutaneous lead delivery system and/or lead increases visibility during and after lead placement resulting in easier lead placement, reduced risk for the patient, improved reliability of lead placement, and avoids improper or premature deployment of lead retention anchors. To create echogenicity in an object or device one must provide a difference between the acoustic impedance of the object and the acoustic impedance of the surrounding medium. In this case, the surrounding medium is biological tissue or fluids such as blood. Surface modification is one means for creating echogenicity.

In one embodiment, the surface of the insertion tool at the distal end or tip may be modified by creating a diffraction gradient composed of grooves diagonally machined into the tip. Other means for modifying the surface include but are not limited to the following: particle blasting to produce a roughened surface; a masking technique to produce depressions; as well as coating the insertion tool tip with an echogenic material.

In another embodiment, echogenic markings may be placed on the lead body to visualize the locations of the electrodes. For example, surface modified metal strips placed near any or all of the electrodes. Means for creating surface modification include but are not limited to the following: a diffraction gradient composed of grooves diagonally machined into the marker; article blasting to produce a roughened surface; a masking technique to produce depressions; as well as coating the insertion tool tip with an echogenic material.

In another embodiment, echogenic location markers may be placed in various places along the lead that are created by impregnating the lead body with ultrasonically reflective particles. The impregnated areas may be adjacent to the distal and/or proximal electrodes as well as any electrode in the array. Additionally, areas of the lead body adjacent to lead anchors may be impregnated with ultrasonically reflective particles. It is understood that the lead anchors may be made echogenic by surface modification of impregnating the lead anchors with ultrasonic particles. The echogenic location markers may be positioned so that when the lead is in the desired position, the echogenic location markers bracket the phrenic nerve.

It is contemplated that the insertion delivery tool 502 may include the external current generator 18. For example, the external current generator 18 may be located in the handle of the insertion tool 502 instead of outside the sterile surgical environment, which may simplify the system and may reduce the possibility of a breach of the sterile environment.

In this configuration, the lead extension 17 may be connected to an electrical connector in the handle of the insertion tool 502 instead of being routed to a location outside of the sterile environment. In other words, locating the external current generator 18 in a handle of the insertion tool 502 does not require the sterile barrier to be pierced by the lead extension 17. It is also contemplated that locating the external current generator 18 in the handle of the insertion tool 502 may allow for the lead 41 to be directly connected to the external current generator 18, thereby eliminating the need for a lead extension 17. It is further contemplated that the insertion tool 502 with the self-contained electrical equipment may be a one-use (or disposable) device.

Also, the insertion tool 502 may be provided in a kit wherein the insertion tool 502 is enclosed in a sterile packaging. The sterile packaging may also include the entire or part of the delivery system 500 (i.e., the insertion tool 502, the stylet 504, the guidewire, the sheath 508, the dilator, and the lead with electrodes). The kit may be a one-use (disposable) kit. Alternatively, the tools in the kit may be reusable in another sterile kit after undergoing a cleaning and sterilizing process.

To confirm that the electrode assembly 15 is positioned over the phrenic nerve 28, the physician may push a button (or actuate another type of user input such as a switch, knob, or voice activation device) on the handle of the insertion tool 502 that actuates a battery powered electronic circuit within the handle to apply electrical power to the electrode via the conductive lead 41. The lead 41 may be releasably attached to the insertion tool 502 and may extend from the handle along the arm of the tool 502 to the distal end and the electrode assembly 15.

An indicator in the form of a light source, audible alert or other indicator mechanism (either part of the insertion tool 502 or separate from the insertion tool 502), may signal to the physician that electrical power is applied to the electrode assembly 15. For example, the indicator may emit a sound that changes frequency, may emit light that changes color, and/or may generate a repeating signal (audible, visual, or tactile) that changes its periodicity depending on the positioning of the electrode assembly 15 relative to the phrenic nerve 28.

### Interconnected Distributed System to Monitor Patient at Rest and During Sleep

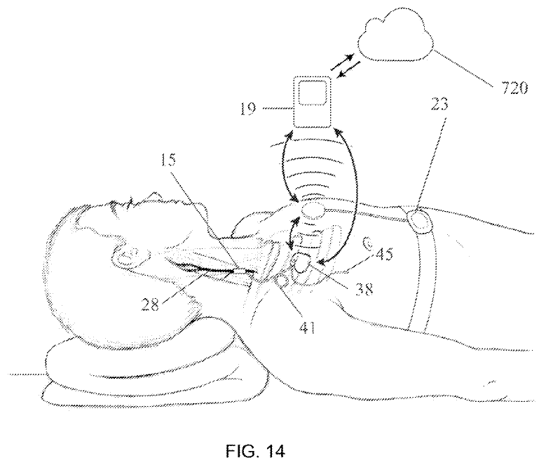

FIG. 14 shows patient in bed during sleep therapy. The patient may be sleeping or resting. The IPG 38 may be implanted in the patient's chest and connected to the electrode assembly 15 by the stimulation lead 41. The electrode assembly 15 may be in electrical contact with the phrenic nerve 28.

The electrode assembly 15 may be an electrode cuff, a paddle electrode, or a lead with electrodes on the surface. The patient may be equipped with a wearable monitoring system 23 in wireless communication with the IPG 38 and the bedside monitor 19 device that can be a MICS communication device removed up to 2.0 meters from the IPG 38. The monitor 19 may be in data communication (e.g., in internet communication) with a cloud computer 720 that enables sharing of data with the physician and the modification of a therapy plan that can be stored in the memory of the IPG 38 and the bedside monitor 19 and executed by the electronics of the IPG 38 in the real time. The bedside monitor 19 may be placed in the pocket of the patient's clothing or placed under the mattress or under the pillow.

The implanted part of the system may include a sensing lead 45 connected to the IPG 38 and tunneled under the skin of the patient's chest to enable improved sensing of thoracic impedance or acceleration from breathing.

An inductive link (shown on FIG. 14 for illustration) may be used to provide communication between a programmer/controller (outside the body) and the implanted device. However, in order to maintain a reliable link, the maximum separation between the two coils, one inside the body and the other outside the body, may be 6 cm or less. Therefore, for an inductive link, the coil outside the body may be kept close to or in contact with the body.

Radar technology may be used as an alternative to an inductive link to conduct non-contact and non-invasive measurements of respiration. Respiratory activity causes a visible and measurable motion to the chest wall. When aiming a radar to a subject's chest, this motion can be recorded and processed to obtain the rates of respiration. Use of radar technology may eliminate the need for both implanted and wearable sensing of respiration. A bedside radar can be integrated into or connected with the bedside monitor 19 and transmit respiratory cycle information to the IPG 38 using MICS communication link in real time. The IPG 38 may apply stimulation energy based on the timing synchronization signals from the bedside monitor 19.

It is appreciated that non-contacting technology for body motion monitoring is rapidly evolving and becoming more advanced and available. For example, U.S. Pat. No. 8,454,528B2 “Non-contact physiologic motion sensors and methods for use” assigned to ResMed Sensor Technologies, Dublin, Ireland describes the use of Doppler-shifted radar signals digitized and processed subsequently to extract information related to the cardiopulmonary motion including respiratory rates, heart rates, waveforms due to respiratory and cardiac activity, direction of arrival, abnormal or paradoxical breathing, etc. In various embodiments, the extracted information can be displayed on a display. It does not teach stimulating a phrenic nerve based on the information collected by Doppler-shifted radar. Specifically, detection of paradoxical movement of the chest wall (where the lung volume may get reduced during the inspiration) can be used as a surrogate variable for inspiratory airway resistance and negative air pressure in the airway distal to the obstruction. This variable may be instrumental in automatic adjustments of energy delivery level or timing for stimulating the phrenic nerve 28.

Once respiratory data from the patient is obtained by an external wearable or non-contacting bedside monitor 19, the respiratory data may be reduced to commands and transmitted to the IPG 38 to act upon the collected data, since the IPG 38 may ultimately control the transmission of stimulation energy to the phrenic nerve 28.

Effective Jan. 14, 2000, the Federal Communications Commission (FCC) established Medical Implant Communications Service (MICS) operating in the 402-405 MHz band. MICS operations may consist of high-speed, ultra-low power, non-voice transmissions to and from implanted medical devices such as cardiac pacemakers and defibrillators. The rules allow use of newly-developed, life-saving medical technology without harming other users of the frequency band. This band has good conductivity in the human body, a higher data rate, and a communication range up to 2 m.

In 2009, the FCC created the Medical Device Radiocommunications Service (MedRadio) in the 401-406 MHz range. The creation of the MedRadio Service incorporated the existing MICS spectrum at 402-405 MHz and added additional spectrum at 401-402 MHz and 405-406 MHz for a total of five megahertz of spectrum for implanted devices as well as devices worn on the patient's body. The communications with the IPG 38 described above may follow MICS and MedRadio rules and standards and may operate in the allocated FCC band and will follow other new and updated standards that will follow MICS.

The maximum allowed energy for each sleeping position for the patient may not be consistent night to night. The allowed energy may be increased or decreased by the physician remotely or automatically by the logic in the IPG 38. On the first night or over several nights after activation, a maximum energy level may be set to some fraction of the maximum tolerable limit determined during the patient's office visit or test night in the sleep lab.

The maximum ramped energy can also be set to some preset fraction of the device operating range that can be for example 1.0 to 5.0 mV. For example, maximum energy level for the first night can be set to 50% of operating range. The next night (e.g., the second night) the energy level may be increased to 51%, the next night after that, the energy level may be increased to 52% followed by further increases of 0.05-5% every night depending on the treatment plan. For example, on the initial night, the increments may be set to a larger value and then gradually decreased following an asymptotic trajectory pseudo-infinitely approaching the maximum value at the diminishing rate. In this way the patient's central nervous system may be expected to gradually adapt to higher energy levels and new, higher tolerability thresholds can be established gradually and without frequent visits to the clinic.

Automatic telemetry can store relevant parameters such as delivered energy and corresponding physiologic respiratory parameters: motion, posture, breathing and oxygenation patterns and communicate these parameters to the physician or the central analytical facility to supervise therapy. A physician may intervene by suspending, reversing, or adjusting, for example slowing down or speeding up the adaptation ramp. There are clear advantages in removing some sensing and control functions from the IPG and redistributing them between outside components communicating wirelessly. The IPG can be made simple, more reliable, smaller with a betterer battery life. The surgical procedure can be simplified. At the same time the quality of the signal may be improved since the IPG has limited access to the patient's respiratory system.

Different embodiments of the novel system for optimization of phrenic nerve stimulation parameters to treat OSA may be a distributed system comprising an IPG in electrical communication with the cervical phrenic nerve and components external to the body. The system is capable of delivering controllable excitatory nerve stimulation pulse trains at precise time intervals where an electrical stimulation causes a diaphragmic contraction of variable strength ranging between twitch capture to fused muscle. In one configuration, the system may further comprise a bedside monitoring controller in continuous wireless communication with the IPG. The bedside controller may be remote to the patient at the distance of more than 6 cm and less than 2 meters. The communication band may be in the 400-410 MHz range. The bedside controller may incorporate a mm wave range radar motion detection device capable of detecting chest motion and estimating breathing rate, separate movement of chest and abdomen, tidal volume and inspiratory time, patients motion, position and blood pulsations and heart rate. This information may be processed by computer logic and used to start, stop, ramp, increase or decrease stimulation energy and adjust stimulation rate.

Paradoxical movement of the chest wall (where the lung volume may get reduced during the inspiration) can be used as a surrogate variable for inspiratory airway resistance and negative air pressure in the airway distal to the obstruction. Paradoxical movement can be explained by the reparatory pump inhaling against the elevated upper airway resistance or closed airway. A large magnitude of negative pressure can be achieved in the chest under this circumstance, especially if patient is hyperventilating in response to increasing blood CO2 and hypoxia. Chest muscles and structure cannot resist this negative pressure and chest paradoxically collapses while the abdomen bulges out. These patterns can be detected by respiratory belts, accelerometers or non-contacting radar-based motion detectors. A paradoxical movement index can be derived. This calculated variable is used to make automatic adjustments of energy delivery level or timing as described.

### Implantable Pulse Generator (IPG) System Design

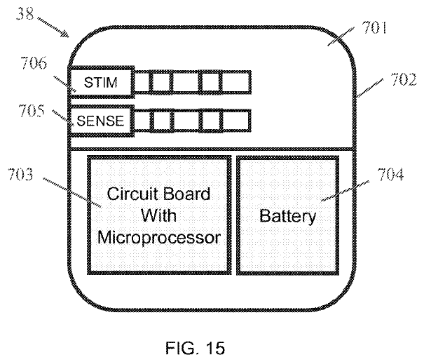

FIG. 15 schematically illustrates an IPG 38 suitable for being implanted in a patient with OSA to stimulate the phrenic nerve. The IPG 38 may include a header 701 for connection to at least one stimulation lead 41 and an optional sensing lead 45, and a hermetically sealed housing 702 for containing an electronic circuit 703 (electronics) and a suitable hermetically sealed battery 704. The battery may be rechargeable using wireless energy transfer. The IPG 38 may contain an accelerometer and/or other sensors such as an oxygen sensor or a vibration or sound sensor (a microphone). Alternatively, or additionally, sensors may be incorporated in a distal portion of the sensing lead 45 or a distal portion of the stimulation lead 41 with the electrical connection to the electronics 703 internal to the IPG 38. The standard implantable connectors may be similar in design and construction to the low-profile IS-1 connector system (per ISO 5841-3) used in cardiac pacemakers. The IS-1 connectors have been in use since the late 1980s and have been shown to be reliable and provide easy release and re-connection over several implantable pulse generator replacements during the service life of a single pacing lead.

According to one technical aspect, the IPG 38 may use a standard, commercially available micro-power, flash (in-circuit programmable) programmable microcontroller or processor core in an application specific integrated circuit (ASIC). This device (or possibly more than one such device for a computationally complex application with sensor input processing) and other large semiconductor components may have custom packaging to reduce circuit board real estate needs.

The IPG 38 may be controlled using microprocessors with the embedded resident operating system software (code). This operating system software may be further broken down into subgroups including system software and application software. The system software may control the operation of the IPG 38 while the application software may interact with the system software to instruct the system software on what actions to take to deliver appropriate amount of energy to the phrenic nerve at the appropriate time.

As shown in FIG. 15, the electronic circuitry 703 contained in the IPG 38 may enable wireless telemetry communication with the external programmer systems and therapy controllers that can be wearable, handheld, or bedside devices, commonly not implanted in the patient. In one embodiment IPG 38 may be responsible for detection of respiration and calculation of respiratory rate via the sensing system, determination of the start time and duration of a stimulation signal, and delivery of a controlled electrical stimulation signal sequence (pulse train) via the stimulation lead 41. In one embodiment, the wearable monitoring system 23 may be in wireless communication with the IPG 38 and the bedside monitor 19 that is in data communication with the cloud computer 720 that enables sharing of data with the physician and modification of therapy plan that can be stored in the memory of IPG 38 and the bedside monitor 19 and executed by the electronics of the IPG 38 in the real time. The IPG 38 may also record and transmit therapy history data (device settings, status, measured data, device use, respiration data, stimulation delivery data, statistics based on motion and sleep time, measured signals, etc.) and implement the patient therapy plan.

As illustrated by FIG. 15, the header 701 may form the top portion of the IPG 38 and may be molded of a polymer that is hermetically sealed to the housing 702. The housing 702 may be a formed titanium casing. As mentioned in the context of respiration sensing, the housing 702 may be used as an electrode for bioimpedance signals including respiration measurement. Similarly, electrode systems on leads may be used as an electrode for bioimpedance respiration measurement. For example, the housing 702 may comprise current emitting and voltage sensing electrodes for respiration detection. Alternatively, separate electrodes may be included in the header 701 of the IPG 38 from which to sense or stimulate.

The header 701 illustrated in FIG. 15 may include two ports: one sensing lead port 705 (labeled “sense”) for receiving the proximal connector of the sensing lead 45 and one stimulation lead port 706 (labeled “stim”) for receiving the proximal connector of the stimulation lead 41. More ports can be added for other leads. The port configured to receive a stimulation lead 41 may include two set screws (labeled “−” for cathode and “+” for anode) with associated set screw blocks and seals for mechanical and electrical connection to corresponding contacts on a commonly used proximal male plug-in connector of the lead. This design and configuration of the lead—header interface may be accepted as standard for devices of this type.

Similarly, the port that is configured to receive a sensing lead may include set screws for current emitting electrodes and voltage sensing electrodes with associated set screw blocks and seals for mechanical and electrical connections to corresponding contacts of the proximal connector of the sensing lead 45. Seals may be located between electrical contacts as well as between the distal-most electrical contact and the remainder of the proximal connector assembly. These seals electrically isolate each contact. The header may also include suture holes for securing the IPG to subcutaneous tissue such as muscle fascia using sutures when implanted in a subcutaneous pocket.

The IPG 38 may generate the stimulation output for delivery to the phrenic nerve 28 by way of the stimulation lead 41. For this purpose, the IPG 38 may have a bipolar stimulation output channel corresponding to the stimulation port, with the channel providing a pulse train of biphasic constant current pulses with a frequency range of 20 to 50 Hz, typically 30 Hz, a pulse width range of 30 to 215 μs, typically 150 μs an amplitude range of 0.4 to 5.0 mA, typically 1.0 to 4.0 mA and a stimulation duty cycle range of 30-70%, typically 40-50% by way of example, not limitation. These ranges may depend on the individual patient and on the configuration of the electrode system such as a nerve cuff or a paddle electrode.

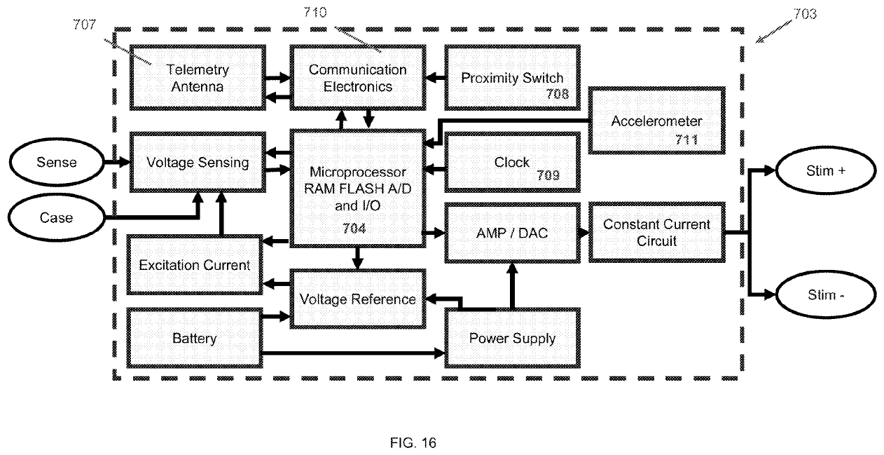

As shown on FIGS. 15 and 16 and in the accompanying description of the IPG, component design, parameters values and component configurations are given by way of illustration, as a possible example of an embodiment, not limitations. Implantable nerve stimulators evolve rapidly, and traditional technologies are expected to give way to battery-less and leadless stimulators. These improvements are designed to reduce size and increase longevity and reliability of stimulators rather than alter their function in a substantive way.

Impedance sensing is optional for some embodiments of the invention. The IPG circuitry may generate the excitation signal and measures voltage by way of the respiration sensing lead 45 for bioimpedance respiration detection. For this purpose, the IPG may have a respiration sensing channel for acquisition of bioimpedance sensing on the desired vector. The vector can be between the sensing lead electrode, the stimulation lead 41 electrode and the casing (case) of the IPG implanted in the chest.

The IPG 38 may measure bioimpedance via the port 705, with the electric connections inside providing a small excitation current (“I”) and measuring voltage (“V”). The excitation signal may comprise a 10 to 50 kHz biphasic constant current pulse, with the positive and negative phases of each biphasic pulse having amplitude of 500 μA. Current (“I”) may be fixed by the circuit, allowing voltage (“V”) to be a relative measure of impedance (“Z”), which corresponds to movement of the muscles, lung, airway, and other structures to produce a signal indicative of respiratory activity.

FIG. 16 schematically illustrates the circuit board 703 that resides in the sealed casing of the IPG 38. A block diagram for this example is shown schematically. The circuit 703 utilizes a microprocessor to control telemetry communications with the external parts of the system, operating the sensing circuits to monitor motion and respiration, controlling the delivery of output stimuli, monitoring the accelerometer, magnetically sensitive proximity sensor (e.g. a reed switch) and the real-time clock. The microprocessor contains built-in support circuits (RAM, flash memory, analog to digital (A/D) converter, timers, serial ports and digital IO) used to interface with the rest of the circuit 703, including the accelerometer 711.

Several dedicated microprocessors communicating via a serial link may be used instead of one microprocessor dividing functions such as stimulation, monitoring and telemetry communications. Alternatively, a single microprocessor could perform these functions.

The telemetry interface circuits may consist of a tuned telemetry coil circuit 707 and a telemetry driver/receiver circuit 710 to allow digitally encoded communication between the external components and the microprocessor. As an alternative to telemetry coils and an inductive link, RF antennae with associated circuitry may be used to establish a RF link to provide for longer distance telemetry. The proximity sensor or switch 708 may provide a means for the IPG to be controlled by using a magnet of a Near Field Communication device (NFC) placed in close proximity. The real-time clock 709 may provide the basic time base (e.g. 768 Hz) for the IPG circuit 703 as well as a clock (year, day, hour, minute, second) which can be used to control the scheduled delivery of therapy. The clock may also be used to time stamp information about the operation of the system that can be recorded on a sleep epoch, hourly, nightly, weekly, or monthly basis.

The bioimpedance respiratory sensing circuit may be comprised of two main parts: the excitation current source (output) and the voltage sensing circuit (input). Respiration can be detected using a 3 or 4-wire impedance measurement circuit. In a 4-wire measurement, an excitation current is driven through a pair of electrodes, and the resulting voltage is measured on a separate pair of electrodes. In one embodiment of a 3-wire measurement, the IPG housing (case) may be used as both an excitation and sensing electrode. The excitation current circuit delivers bursts of biphasic pulses of low level (e.g. 450 uA) current to the selected electrode pair every 100 ms during sensing. The voltage sensing amplifier circuit synchronously monitors the voltage produced by the excitation current on the selected electrode pair. The resulting output signal is proportional to the respiratory impedance (0.072 to 100) and is applied to the A/D circuit in the microprocessor for digitization and analysis. Other sensing circuits can include an ECG signal amplifier (not shown but well known from cardiac pacemakers) or a pulse oximetry interface.

The stimulation output circuits may deliver bursts of biphasic stimulation pulses to the stimulation lead 41. These bursts may be synchronized to the sensed respiratory waveform to deliver stimulation and thus generating negative airway pressure and reflex causing airway opening at the appropriate time. The stimulation output circuits may include an electrode polarity switching network, a current source circuit, and an output power supply. The electrode switching network allows for a charge balancing cycle following each stimulation pulse during which the outputs are connected together with no applied output pulse. The timing and polarity of the pulse delivery is provided by control outputs of the microprocessor. The microprocessor selects the amplitude (e.g. 0.4 mA to 5 mA) of the output current from the current source circuit which is applied through the switching/pulse shaping network. The output power supply converts battery voltage to a higher voltage (e.g. 5V to 15V) which is sufficient to provide the selected current into the load impedance of the lead electrode system, which can be a bipolar system or a monopolar system with the IPG casing used as current return electrode. The microprocessor may measure the voltage output from the electrode resulting from the delivered current and the load impedance. The microprocessor divides the output voltage by the output current resulting in a measure of the load impedance (e.g. 400 Ω to 2800 Ω) which can be an indicator of integrity of the lead electrode system and the conditions of the surrounding tissues.

The system may comprise a rechargeable battery (not shown) and an external controller including charging circuitry, a rechargeable battery coupled to the circuitry, and the circuitry adapted for wireless telemetry and energy transfer, and a charging coil coupled to the external controller for generating the radio frequency magnetic field to transcutaneously recharge the rechargeable battery. During a rechargeable battery recharge period, the external controller may be adapted to be carried by a user with no connection to a power main to allow the user to be completely mobile. Rechargeable IPG batteries and circuits are well understood and available from OEMs; they have certain advantages and disadvantages and are often an issue of preference, rather than necessity.

The IPG (or lead connected to the IPG) may contain an oxygen sensor to monitor oxygen levels, for example during a night therapy session. This signal may be used to monitor efficacy as well to change stimulation delivery settings during a therapy session. For example, the IPG may be programmed to increase stimulation when oxygen de-saturations are detected at a programmable threshold rate and/or severity. In addition, the IPG may turn stimulation on once de-saturations are detected, wherein thresholds of rate and severity are programmable. Desaturations may act to indicate the sleep state or wakefulness. In a similar manner, electroneurogram (ENG) may be used to monitor nerve activity, which may also be indicative of sleep state and/or wakefulness. EMG can be used to monitor muscle activity that may be indicative of spontaneous respiratory effort. The IPG may use the indication of sleep state or wakefulness to change stimulation settings. For example, stimulation may be increased when the patient is estimated to likely be in the N3 or REM sleep. Stimulation level may be decreased or turned off during stage N1 or wakefulness.

As illustrated by FIG. 14, all sensors need not be implanted in the patent's IPG and implanted sensors electrically connected to the IPG but can be distributed between the internal and external components of the system. Modern wireless communication between the IPG pulse generator and external devices can be short distance radio communications. The wireless telemetry communication between the external controller and the pulse generator may operate in the MICS (Medical Implant Communications Service) band at between about 402 MHz to about 405 MHz as described above.

The IPG circuitry may contain a three-axis accelerometer 711 that can be used to determine the patient's body position (supine, prone, upright, left, or right side) and/or detect motion events (wakefulness). These data may be used to change stimulation settings or inhibit output. The IPG may be programmed to increase stimulation intensity when the patient is in specific body positions (e.g., supine, a more challenging position). The IPG may segregate recorded therapy statistics (e.g., cycling detector events, oxygen desaturations) with respect to body position. For example, a patient's cycling detector may record very few events in the lateral position and many events in the supine position, indicative of the patient being treated in the lateral position.

The bioimpedance respiration signal (“Z”) is generated by dividing the change in measured voltage (“V”) by the excitation current (“I”). It may index diaphragm movement, expansion and contraction of a lung and airway over time and therefore is an accepted good measure of respiratory activity. It may be used to estimate in real time, with known imperfections, respiratory effort, respiratory rate, respiratory (tidal) volume, minute volume, etc. If the excitation current (I) is constant or assumed constant, then the bioimpedance (Z) is proportional to the measured voltage (V), and thus the voltage (V) may be used as a surrogate for bioimpedance (Z), thereby eliminating the division step. As used in this context, diaphragm movement includes movements and shape changes of the diaphragm, lung, large airways, and adjacent tissue that occur during normal breathing and during obstructed breathing. The bioimpedance waveform may be filtered to reduce noise and eliminate cardiac artifact, clarifying positive and negative, expiration and inspiration peak occurrence. The signal may be filtered using a first order low pass filter. Alternatively, a higher order filtering approach could be utilized to filter the signal. The (positive or negative) peak of the impedance signal corresponds to the end of the inspiratory phase and the beginning of the expiratory phase. If the signal is normal, the positive peak is used; and if the signal is inverted, the negative peak is used. The beginning of the inspiratory phase occurs somewhere between the peaks and may not be readily discernable. The impedance signal typically provides a reliable timing event for end-inspiration and begin-expiration event. The rest of the breathing cycle may need to be extrapolated based on the patient's history.

Body motion is often indicative of patient wakefulness, can be detected by the accelerometer and may also change the bioimpedance signal (Z). Different thresholds of sensitivity may be utilized such that minor movements are not confused with major motion events such as rolling from side to back in bed, sitting up, standing up or walking around. When a motion event is determined, stimulation may be turned off or turned down until motion stops or for a programmable duration of time. The frequency and duration of these motion events may be recorded in device history. The accelerometer could be utilized in a similar fashion alone or in combination with impedance to detect and record motion events.

Waxing and waning of the bioimpedance signal (Z) is often indicative of apneas or hypopneas. Generally referred to as cycling, this pattern may be detected, for example, by assessing trends of increasing and decreasing average P-P amplitude values. Different thresholds of sensitivity may be utilized such that minor changes in P-P values are not declared cycling events. When cycling is detected, stimulation parameters may be initiated or changed (e.g., increased intensity, increased duty cycle, etc.) to improve therapy. The frequency and duration of these cyclic breathing patterns may be recorded in therapy history. These values may be used as an indicator of how well the patient is being treated, providing an estimate of AHI.

The IPG may be programmed to change stimulation level between therapy sessions, days, or other programmable value. The stimulation level may be recorded alongside therapy session data, for example cycling rate, oxygen desaturation frequency and severity, stimulation time, variations in respiratory rate, variations in respiratory prediction, etc. processor 703 is equipped with different computer memory types that can store program code, settings, and patient data in areas with different electronic ways and speeds of updating the contents of memory.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both. Furthermore, characteristics or steps which have been described may also be used in combination with other characteristics or steps and in any order unless the disclosure or context suggests otherwise.

1. A method for inserting and securing an implantable electrode lead assembly configured to be positioned in a patient's neck to stimulate a phrenic nerve comprising the steps of:
generating an ultrasound image of internal tissues within the patient's neck at the C7 level;
inserting a flexible lead stiffened by a stylet along a fascia defining the boundary of the patient's sternocleidomastoideole and the patient's anterior scalene muscle under in-plane, using real time ultrasound guidance, said flexible lead including a plurality of electrodes and echogenic markers located at a distal end of the lead;
positioning the electrodes within the patient's neck so that the echogenic markers bracket the patient's phrenic nerve;
applying an electric current to elicit a diaphragmatic contraction to confirm electrode proximity to the phrenic nerve;
repositioning the lead if the electric current is higher than an acceptable stimulation current threshold;
securing the lead by deploying securement devices; and
removing the stylet.

2. The method of claim 1, wherein the internal tissues include at least one of patient's brachial plexus, sternocleidomastoideole and the anterior scalene muscle, and phrenic nerve.

3. The method of claim 1, further comprising inserting an insertion tool to create a path for implanting the flexible lead.

4. The method of claim 1, wherein the securement devices comprise resilient elements and the step of securing the lead comprises deploying the resilient elements into the patient's sternocleidomastoideole and anterior scalene muscle.

5. The method of claim 1, wherein deployment of the securement devices activates the securement devices from a collapsed state to an expanded state.

6. The method of claim 1, wherein repositioning the lead includes realigning the echogenic markers relative to the phrenic nerve.

7. The method of claim 1 further comprising the steps of:
creating strain relief and suturing the lead to the fascia at an insertion site;
creating an implantable pulse generator (IPG) pocket below the collarbone of the patient by creating an incision large enough to allow the placement of an implantable pulse generator (IPG) and using blunt dissection;
placing the IPG inside the IPG pocket;
subcutaneously tunneling said lead from an electrode implant site to the IPG pocket; and
inserting a proximal part of said lead into a header of the IPG, said proximal part of said lead being a connector configured to deliver electrical stimulation generated by the IPG to the electrodes.

8. The method of claim 7, further comprising the steps of:
applying an electric current to elicit a diaphragmatic contraction to confirm electrode proximity to the phrenic nerve;

9. A method for guiding a nerve stimulation therapy delivery device to a cervical phrenic nerve of a patient comprising the steps of:
selecting a target section of the phrenic nerve;
generating an ultrasound image that identifies the anatomic landmarks, the ultrasound image being generated by obtaining a short axis ultrasound image at the root of the patient's neck at the C7 level;
identifying a predetermined implantation pathway that defines a trajectory that avoids predetermined anatomic elements and extends between an insertion point under the patient's platysma at the edge of the sternocleidomastoideole and terminates at a target location on a surface of the patient's anterior scalene muscle;
inserting an insertion tool into the insertion point after the implantation pathway has been identified;
navigating the insertion tool through the predetermined implantation pathway, under real time ultrasound guidance, until a distal tip of the insertion tool is positioned adjacent or proximate to the target section of the nerve;
advancing a therapy delivery device, under ultrasound guidance, through the introducer to the target section of the nerve;
applying an electric current to elicit a diaphragmatic contraction and confirm therapy delivery device proximity to the phrenic nerve;
repositioning the therapy delivery device if the electric current is higher than an acceptable stimulation current threshold; and
securing the therapy delivery device in place by deploying securement devices.

10. The method of claim 9, wherein the insertion point is on the surface of the patient's skin or under the patient's skin.

11. The method of claim 9, wherein said anatomical landmarks include at least one of the patient's brachial plexus, carotid artery, and sternocleidomastoideole.

12. The method of claim 9, further comprising the steps of:
creating strain relief and suturing to the patient's fascia at the insertion point;
creating an implantable pulse generator (IPG) pocket below the patient's collarbone by creating an incision large enough to allow the placement of the IPG and using blunt dissection;
subcutaneously tunneling said the therapy delivery device from the target section of the nerve to the IPG pocket;
inserting a proximal part of said therapy delivery device into a header of the IPG, said proximal part of said therapy delivery device being a connector configured to deliver electrical stimulation generated by the IPG to electrodes located at a distal end of the therapy delivery device;
applying another electric current to elicit another diaphragmatic contraction and reconfirm electrode proximity to the phrenic nerve;
placing the IPG inside the IPG pocket then applying another electric current to elicit another diaphragmatic contraction to reconfirm placement of the therapy delivery device and confirm electrode impedance and a status of a battery of the IPG;
closing the incision site after the electrode impedance and IPG battery status are deemed acceptable.

13. A nerve stimulation lead assembly with an anchoring system, the nerve stimulation lead assembly comprising:
a lead body with a proximal end configured to connect to an electrical power source and a distal end configured to be positioned adjacent a patient's phrenic nerve;
at least one electrode positioned at the distal end of the lead body;
a first anchor positioned between the at least one electrode and a terminus of the distal end of the lead body;
a sheath that encloses the lead body and is axially movable relative to the lead body,
wherein the first anchor is biased toward an extended position in which the first anchor extends radially outwardly from the lead body, and
wherein the sheath is configured so that when the sheath covers the first anchor, the first anchor is kept in a retracted position against a surface of the lead body and when the sheath does not cover the first anchor, the first anchor is in the extended position.

14. The nerve stimulation lead assembly of claim 10, further comprising a second anchor on an opposite side of the at least one ele3trode from the first anchor.

15. The nerve stimulation lead assembly of claim 14, wherein the second anchor is biased toward an extended position in which the second anchor extends radially outwardly from the lead body, and
wherein the sheath is configured so that when the sheath covers the second anchor, the second anchor is kept in a retracted position against a surface of the lead body and when the sheath does not cover the second anchor, the second anchor is in the extended position.

16. The nerve stimulation lead assembly of claim 14, wherein the sheath is configured so that the first anchor is deployable to the extended position without deploying the second anchor to the extended position.

17. The nerve stimulation lead assembly of claim 14, wherein the first and second anchors are configured to anchor into tissue adjacent to the nerve stimulation lead assembly to resist axial movement of the lead body.

18. The nerve stimulation lead assembly of claim 14, wherein the first and second anchors comprise radially extending tines, curling tines, and/or barbs.

19. The nerve stimulation lead assembly of claim 14, wherein the first and second anchors are formed from a shape memory alloy.

20.-120. (canceled)
